(19) United States
(12) Patent Application Publication
(10) Pub. No.: US 2024/0127450 A1
(43) Pub. Date: Apr. 18, 2024

(54) MEDICAL IMAGE PROCESSING APPARATUS AND NON-TRANSITORY COMPUTER READABLE MEDIUM

(71) Applicant: CANON MEDICAL SYSTEMS CORPORATION, Tochigi (JP)

(72) Inventors: Saki YOSHIDA, Shimoda (JP); Takuya SAKAGUCHI, Utsunomiya (JP)

(73) Assignee: CANON MEDICAL SYSTEMS CORPORATION, Tochigi (JP)

(21) Appl. No.: 18/486,415

(22) Filed: Oct. 13, 2023

(30) Foreign Application Priority Data
Oct. 14, 2022 (JP) 2022-165724

(51) Int. Cl. G06T 7/00; A61B 6/00; G16H 30/40

(52) U.S. Cl. CPC G06T 7/0016; A61B 6/504; A61B 6/5217; G16H 30/40; G06T 2207/10081; G06T 2207/30104

(57) ABSTRACT

A medical image processing apparatus according to an embodiment includes processing circuitry configured to acquire geometric data indicating a position of a region of interest set on a first blood vessel image, specify a corresponding region in a second blood vessel image that corresponds to the region of interest on the basis of the geometric data, and acquire information on the corresponding region.

### CROSS-REFERENCE TO RELATED APPLICATIONS

This application is based upon and claims the benefit of priority from Japanese Patent Application No. 2022-165724, filed on Oct. 14, 2022; the entire contents of which are incorporated herein by reference.

### FIELD

Embodiments disclosed herein generally relate to a medical image processing apparatus and non-transitory computer readable medium.

### BACKGROUND

In the related art, in order to assist in the formulation and the like of the diagnosis and treatment plans of diseases related to blood vessels, technologies are known to acquire medical images in which blood vessels are depicted using a contrast agent or acquire an index related to blood flow, and to present the images and the index to users such as physicians. The user makes a diagnosis by paying particular attention to a region of interest. Examples of the region of interest include regions including a suspected lesion or treatment target regions.

### BRIEF DESCRIPTION OF THE DRAWINGS

FIG. 1 is a block diagram illustrating an example of the configuration of a medical image diagnostic system according to a first embodiment;

FIG. 2A is a block diagram illustrating an example of the configuration of an X-ray diagnostic apparatus according to the first embodiment;

FIG. 2B is a diagram illustrating a biplane X-ray diagnostic apparatus according to the first embodiment;

FIG. 3 is a block diagram illustrating an example of the configuration of an X-ray CT apparatus according to the first embodiment;

FIG. 4 is a block diagram illustrating an example of the configuration of a medical image processing apparatus according to the first embodiment;

FIG. 5 is a flowchart illustrating a series of processes according to the first embodiment;

FIG. 6 is a diagram for explaining the specifying of a corresponding region according to the first embodiment.

FIG. 7A is a display example according to the first embodiment;

FIG. 7B is a display example according to the first embodiment; and

FIG. 7C is a display example according to the first embodiment.

### DETAILED DESCRIPTION

A medical image processing apparatus according to the embodiment comprises processing circuitry configured to acquire geometric data indicating a position of a region of interest set on a first blood vessel image, specify a corresponding region in a second blood vessel image that corresponds to the region of interest on a basis of the geometric data, and acquire information on the corresponding region.

Embodiments of the medical image processing apparatus and non-transitory computer readable medium are described in detail below with reference to the accompanying drawings.

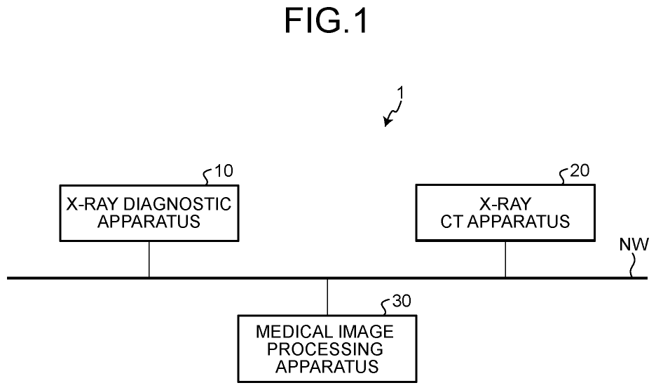

In the present embodiment, a medical image diagnostic system 1 including an X-ray diagnostic apparatus 10, an X-ray computed tomography (CT) apparatus 20, and a medical image processing apparatus 30 is described as an example. FIG. 1 is a block diagram illustrating an example of the configuration of the medical image diagnostic system 1 according to the first embodiment.

The X-ray diagnostic apparatus 10, the X-ray CT apparatus 20, and the medical image processing apparatus 30 are interconnected via a network NW. The X-ray diagnostic apparatus 10, the X-ray CT apparatus 20, and the medical image processing apparatus 30 can be installed at any location as long as they can be interconnected via the network NW. For example, the medical image processing apparatus 30 may be installed in hospitals or other facilities differently from the X-ray diagnostic apparatus 10 and the X-ray CT apparatus 20. That is, the network NW may be configured by a local network closed within the hospital, or may be a network via the Internet.

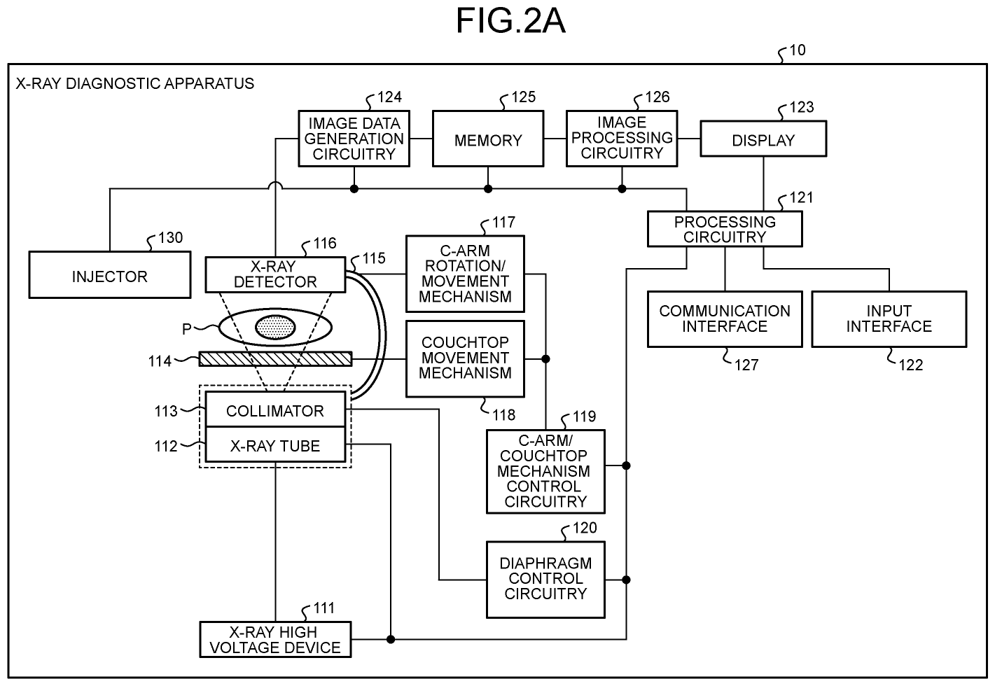

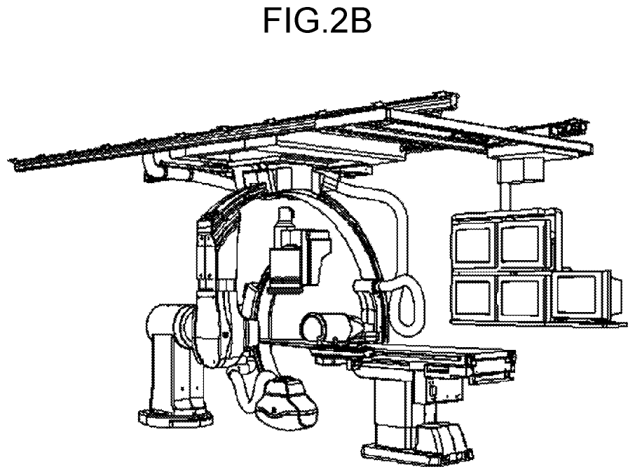

A configuration example of the X-ray diagnostic apparatus 10 is described with reference to FIG. 2A. FIG. 2A is a block diagram illustrating an example of the configuration of the X-ray diagnostic apparatus 10 according to the first embodiment. For example, the X-ray diagnostic apparatus 10 includes an X-ray high voltage device 111, an X-ray tube 112, a collimator 113, a tabletop 114, a C-arm 115, an X-ray detector 116, a C-arm rotation/movement mechanism 117, a tabletop movement mechanism 118, C-arm/tabletop mechanism control circuitry 119, diaphragm control circuitry 120, processing circuitry 121, an input interface 122, a display 123, image data generation circuitry 124, a memory 125, image processing circuitry 126, a communication interface 127, and an injector 130.

The X-ray high voltage device 111 generates a high voltage and applies the high voltage to the X-ray tube 112. The X-ray tube 112 is a vacuum tube with a cathode (filament) that generates thermal electrons and an anode (target) that generates X-rays upon impact of the thermal electrons. The X-ray tube 112 generates X-rays by emitting thermal electrons from the cathode to the anode by using the high voltage applied from the X-ray high voltage device 111. The collimator 113 is an X-ray diaphragm that narrows down an emission range of the X-rays generated by the X-ray tube 112. For example, the collimator 113 includes a plurality of diaphragm blades each made of lead or the like, and forms an irradiation port. The tabletop 114 is a bed on which a subject P is placed.

The X-ray detector 116 detects X-rays emitted from the X-ray tube 112 and transmitted through the subject P, and outputs a detection signal corresponding to the detected X-ray dose. For example, the x-ray detector 116 is an x-ray flat panel detector (FPD) having detection elements arranged in a matrix form. The C-arm 115 holds the x-ray tube 112 and the x-ray detector 116 facing each other with the subject P interposed between the x-ray tube 112 and the x-ray detector 116.

The C-arm rotation/movement mechanism 117 controls the rotation and movement of the C-arm 115 by using power generated by an actuator, under the control of the C-arm/tabletop mechanism control circuitry 119. For example, the C-arm rotation/movement mechanism 117 changes an imaging angle by rotating the C-arm 115. The C-arm rotation/movement mechanism 117 also changes an imaging position by moving the C-arm 115.

The tabletop movement mechanism 118 moves the tabletop 114 by using the power generated by the actuator, under the control of the C-arm/tabletop mechanism control circuitry 119. For example, the tabletop movement mechanism 118 raises and lowers the tabletop 114 when the subject P is placed on the tabletop 114 or is unloaded from the tabletop 114. The tabletop movement mechanism 118 can also change the imaging position by moving the tabletop 114 and changing the position of the tabletop 114 relative to the C-arm 115.

The diaphragm control circuitry 120 controls the emission range of the X-rays by controlling the operation of the collimator 113. For example, the diaphragm control circuitry 120 slides the diaphragm blades of the collimator 113, thereby controlling the shape and size of the irradiation port and narrowing down the emission range of the X-rays generated by the X-ray tube.

The input interface 122 receives various input operations from a user, converts the received input operations into electrical signals, and outputs the electrical signals to the processing circuitry 121. For example, the input interface 122 is implemented by a mouse, a keyboard, a trackball, a switch, a button, a joystick, a touch pad for performing an input operation by touching an operation surface, a touch screen with integrated display screen and touch pad, non-contact input circuitry using an optical sensor, voice input circuitry, or the like. The input interface 122 may be configured as a tablet terminal or the like capable of wirelessly communicating with the processing circuitry 121. The input interface 122 may also be circuitry that receives input operations from the user through motion capture. In one example, the input interface 122 can receive the body movement, line of sight, or the like of the user as input operations by processing signals acquired via a tracker and images acquired about the user. The input interface 122 is not limited to only those with physical operation components such as a mouse and a keyboard. For example, an example of the input interface 122 also includes electrical signal processing circuitry that receives an electrical signal corresponding to an input operation from an external input device provided separately from the X-ray diagnostic apparatus 10 and outputs the electrical signal to the processing circuitry 121.

The display 123 displays various information under the control of the processing circuitry 121. For example, the display 123 displays a graphical user interface (GUI) for receiving various instructions, settings, and the like from the user via the input interface 122. The display 123 also displays X-ray images taken. For example, the display 123 is a liquid crystal display or a cathode ray tube (CRT) display. The display 123 may be a desktop type or may be configured as a tablet terminal or the like capable of wirelessly communicating with the processing circuitry 121.

The image data generation circuitry 124 generates projection data by using the detection signal output from the X-ray detector 116, and stores the generated projection data in the memory 125. For example, the image data generation circuitry 124 generates projection data by performing current/voltage conversion, analog (A)/digital (D) conversion, and parallel/serial conversion on the detection signal received from the X-ray detector 116. Then, the image data generation circuitry 124 stores the generated projection data in the memory 125.

The memory 125 is implemented by a semiconductor memory element such as a random access memory (RAM) and a flash memory, a hard disk, an optical disk, or the like. For example, the memory 125 stores X-ray images of the subject P that have been taken. The memory 125 also stores computer programs for causing the circuitry included in the X-ray diagnostic apparatus 10 to implement the functions thereof. The memory 125 may be implemented by a group of servers (cloud) connected to the X-ray diagnostic apparatus 10 via the network NW.

The image processing circuitry 126 generates X-ray images by performing various types of image processing on the projection data generated by the image data generation circuitry 124, under the control of the processing circuitry 121. For example, the image processing circuitry 126 performs various processes by using image processing filters such as moving average (smoothing) filters, Gaussian filters, median filters, recursive filters, and bandpass filters. The image processing circuitry 126 may store the generated X-ray images in the memory 125.

The communication interface 127 consists of, for example, a network card, a network adapter, or the like. The communication interface 127 transmits and receives various information to and from devices connected via the network NW, under the control of the processing circuitry 121.

The injector 130 controls the injection of a contrast agent into the blood vessels of the subject P. In the X-ray diagnostic apparatus 10, X-rays are emitted in a state in which a contrast agent is injected into the blood vessels, so that X-ray images depicting the blood vessels can be acquired. That is, the X-ray diagnostic apparatus 10 is an X-ray angiography apparatus used for diagnosing the blood vessels.

For example, the X-ray diagnostic apparatus 10 acquires X-ray images while injecting a contrast agent and presents the X-ray images to a user such as a physician during a procedure such as cardiac percutaneous coronary intervention (PCI). That is, the X-ray diagnostic apparatus 10 acquires blood vessel images and presents the blood vessel images to the user. This allows the user such as a physician to proceed with a procedure while confirming a device inserted into the body of the subject P on the X-ray image.

The X-ray diagnostic apparatus 10 can also generate a digital subtraction angiography (DSA) image with background components such as soft tissue and bone removed by performing difference between an X-ray image taken with a contrast agent injected (contrast image) and an X-ray image taken without a contrast agent injected (mask image), and present the DSA image to the user. In the following, contrast images, DSA images, and the like are simply referred to as blood vessel images without any particular distinction. That is, regardless of whether image processing such as difference processing from a mask image is performed, an image in which blood vessels appear is referred to as a blood vessel image. The same is true for blood vessel images acquired by the X-ray CT apparatus 20 to be described below.

For example, the X-ray diagnostic apparatus 10 acquires blood vessel images prior to a procedure such as cardiac PCI and presents the blood vessel images to a user such as a physician. This allows the user such as a physician to diagnose the presence or absence and the condition of a lesion, and to make a treatment plan if necessary.

The type of contrast agent is not particularly limited, and may be a positive contrast agent consisting mainly of iodine, barium sulfate, or the like, or a gaseous contrast agent such as carbon dioxide. The contrast agent may be automatically injected by the injector 130 or manually by the user such as a physician.

The X-ray diagnostic apparatus 10 may be a biplane apparatus as illustrated in FIG. 2B. That is, the X-ray diagnostic apparatus 10 may include two sets of imaging mechanisms each consisting of the X-ray tube 112, the collimator 113, the tabletop 114, and the C-arm 115. In this case, the X-ray diagnostic apparatus 10 can substantially simultaneously capture images of blood vessels from two directions, and present the images to the user such as a physician.

FIG. 2A is merely an example, and the specific configuration can be changed as appropriate. For example, respective processing functions of the processing circuitry 121 may be implemented by being distributed to a plurality of pieces of processing circuitry. Various pieces of circuitry such as the C-arm/tabletop mechanism control circuitry 119, the diaphragm control circuitry 120, the processing circuitry 121, the image data generation circuitry 124, and the image processing circuitry 126 may be integrated and implemented as appropriate.

The various pieces of circuitry in FIG. 2A may also implement functions by using a processor in an external device connected via the network NW. For example, the processing circuitry 121 implements functions by reading and executing computer programs corresponding to respective functions from the memory 125, and using, as computing resources, a group of servers (cloud) connected to the X-ray diagnostic apparatus 10 via the network NW.

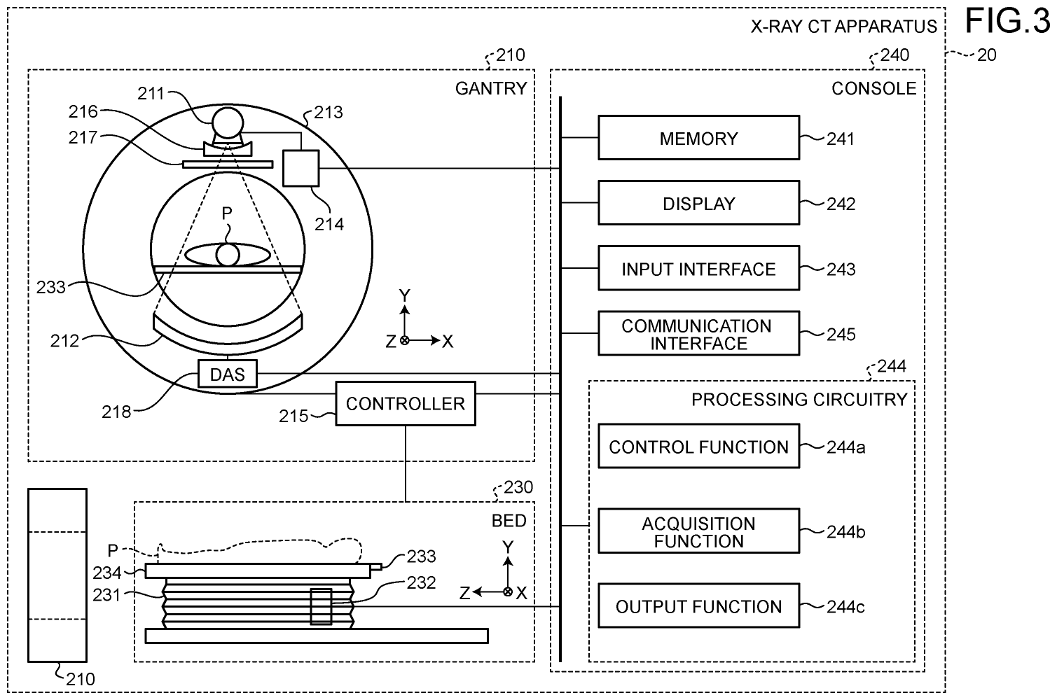

An example of the configuration of the X-ray CT apparatus 20 is described below with reference to FIG. 3. FIG. 3 is a block diagram illustrating an example of the configuration of the X-ray CT apparatus 20 according to the first embodiment. For example, the X-ray CT apparatus 20 includes a gantry 210, a bed 230, and a console 240.

In FIG. 3, the longitudinal direction of a rotation axis of a rotating frame 213 or a tabletop 233 of the bed 230 in a non-tilted state is referred to as a Z-axis direction. An axis direction orthogonal to the Z-axis direction and horizontal to a floor surface is referred to as an X-axis direction. An axis direction orthogonal to the Z-axis direction and perpendicular to the floor surface is referred to as a Y-axis direction. FIG. 3 illustrates the gantry 210 drawn from a plurality of directions for description, and a case where the X-ray CT apparatus 20 has one gantry 210.

The gantry 210 includes an X-ray tube 211, an X-ray detector 212, the rotating frame 213, an X-ray high voltage device 214, a controller 215, a wedge 216, a collimator 217, and a data acquisition system (DAS) 218.

The X-ray tube 211 is a vacuum tube with a cathode (filament) that generates thermal electrons and an anode (target) that generates X-rays upon impact of the thermal electrons. The X-ray tube 211 generates X-rays that are emitted to the subject P by thermal electrons emitted from the cathode to the anode due to the application of a high voltage from the X-ray high voltage device 214.

The X-ray detector 212 detects X-rays emitted from the X-ray tube 211 and passing through the subject P, and outputs a signal corresponding to the detected X-ray dose to the DAS 218. The X-ray detector 212, for example, has a plurality of detection element arrays each including a plurality of detection elements arranged in a channel direction along one circular arc centered at the focus of the X-ray tube 211. The X-ray detector 212, for example, has a structure in which a plurality of detection element arrays are arranged in a row direction (slice direction), the plurality of detection element arrays each including the plurality of detection elements arranged in the channel direction.

The rotating frame 213 is an annular frame that supports the X-ray tube 211 and the X-ray detector 212 facing each other and rotates the X-ray tube 211 and the X-ray detector 212 by the controller 215. For example, the rotating frame 213 is a casting made of aluminum. In addition to the X-ray tube 211 and the X-ray detector 212, the rotating frame 213 can further support the X-ray high voltage device 214, the wedge 216, the collimator 217, the DAS 218, and the like. In the following, the rotating frame 213 and a portion that rotationally moves with the rotating frame 213 in the gantry 210 are also referred to as a rotating portion (rotor). A non-rotating portion of the gantry 210 is also referred to as a fixed portion (stator). The fixed portion supports the rotating portion.

The controller 215 controls the operations of the gantry 210 and the bed 230 by using the power generated by the actuator. The wedge 216 is an x-ray filter for adjusting the amount of X-rays emitted from the X-ray tube 211. The collimator 217 is an X-ray diaphragm that narrows down an emission range of the X-rays transmitted through the wedge 216.

The DAS 218 acquires X-ray signals detected by each of the detection elements included in the X-ray detector 212. For example, the DAS 218 has an amplifier that performs an amplification process on electrical signals output from each detection element and an A/D converter that converts the electrical signals into digital signals, and generates detection data. The DAS 218 is implemented by, for example, a processor.

The bed 230 is a device for placing and moving the subject P to be subjected to CT scanning, and includes a base 231, a couch drive device 232, the tabletop 233, and a support frame 234. The base 231 is a housing that supports the support frame 234 in a vertically movable manner. The couch drive device 232 is a drive mechanism that moves the tabletop 233 with the subject P placed thereon in the direction of a long axis of the tabletop 233, and includes a motor, an actuator, and the like. The tabletop 233 provided on an upper surface of the support frame 234 is a board on which the subject P is placed. In addition to the tabletop 233, the couch drive device 232 may move the support frame 234 in the direction of the long axis of the tabletop 233.

The console 240 includes a memory 241, a display 242, an input interface 243, processing circuitry 244, and a communication interface 245. The console 240 is described as being provided separately from the gantry 210, but the gantry 210 may include the console 240 or a part of the components of the console 240.

Details of the memory 241, the display 242, the input interface 243, and the communication interface 245 are not particularly limited; however, for example, the memory 241, the display 242, the input interface 243, and the communication interface 245 can be configured in the same manner as the memory 125, the display 123, the input interface 122, and the communication interface 127 described above.

The processing circuitry 244 controls the overall operation of the X-ray CT apparatus 20 by performing a control function 244a, an acquisition function 244b, and an output function 244c. For example, the processing circuitry 244 serves as the control function 244a by reading a computer program corresponding to the control function 244a from the memory 241 and executing the read computer program. In the same manner, the processing circuitry 244 serves as the acquisition function 244b and the output function 244c. For example, the control function 244a controls the acquisition function 244b and the output function 244c according to instructions from a user received via the input interface 243.

The acquisition function 244b performs a CT scan on the subject P and acquires detection data. For example, the acquisition function 244b supplies a high voltage to the X-ray tube 211 by controlling the X-ray high voltage device 214. This causes the X-ray tube 211 to generate X-rays to be emitted to the subject P. The acquisition function 244b also moves the subject P into an imaging port of the gantry 210 by controlling the couch drive device 232. The acquisition function 244b controls the distribution of X-rays to be emitted to the subject P by adjusting the position of the wedge 216 and the aperture and position of the collimator 217. The acquisition function 244b also rotates the rotating portion by controlling the controller 215. While the CT scan is performed by the acquisition function 244b, the DAS 218 acquires X-ray signals from each detection element in the X-ray detector 212 and generates detection data.

The acquisition function 244b performs preprocessing on the acquired detection data. For example, the acquisition function 244b performs preprocessing such as logarithmic transformation, offset correction, sensitivity correction between channels, and beam hardening correction on the detection data. The data after the preprocessing is also referred to as raw data. The detection data before the preprocessing and the raw data after the preprocessing are also collectively referred to as projection data. The acquisition function 244b generates X-ray CT images (volume data) on the basis of the projection data. For example, the acquisition function 244b generates X-ray CT images by performing a reconstruction process using a filtered back projection method, a successive approximation reconstruction method, or the like on the preprocessed projection data.

The acquisition function 244b can acquire X-ray CT images depicting blood vessels by performing a CT scan with a contrast agent injected into the blood vessels. That is, the acquisition function 244b can acquire a three-dimensional blood vessel image. The contrast agent can be automatically injected, for example, by a device similar to the injector 130 described above. That is, the X-ray CT apparatus 20 is a CT angiography (CTA) apparatus used for diagnosing blood vessels. For example, the X-ray CT apparatus 20 acquires X-ray CT images for a follow-up observation after a procedure such as cardiac PCI is performed.

The output function 244c controls the output of various data. For example, the output function 244c controls display on the display 242. For example, the output function 244c converts the X-ray CT image into a display image, such as an arbitrary cross-sectional image or a rendered image of an arbitrary viewpoint direction, on the basis of input operations received from the user via the input interface 243, and displays the display image on the display 242.

In the X-ray CT apparatus 20 illustrated in FIG. 3, the processing functions are stored in the memory 241 in the form of computer programs executable by a computer. The processing circuitry 244 is a processor that reads the computer programs from the memory 241 and executes the read computer programs, thereby implementing functions corresponding to the executed computer programs. In other words, the processing circuitry 244 in the state of having read the computer programs has functions corresponding to the read computer programs.

In FIG. 3, the control function 244a, the acquisition function 244b, and the output function 244c are described as being implemented by the single processing circuitry 244; however, the processing circuitry 244 may be configured by combining a plurality of independent processors and respective processors may implement functions by executing computer programs. Furthermore, the respective processing functions of the processing circuitry 244 may be implemented by being appropriately distributed or integrated into single processing circuitry or a plurality of pieces of processing circuitry.

The processing circuitry 244 may also implement functions by using a processor of an external device connected via the network NW. For example, the processing circuitry 244 reads computer programs corresponding to respective functions from the memory 241, executes the read computer programs, and uses, as computing resources, a group of servers (cloud) connected to the X-ray CT apparatus 20 via the network NW, thereby implementing the respective functions illustrated in FIG. 3.

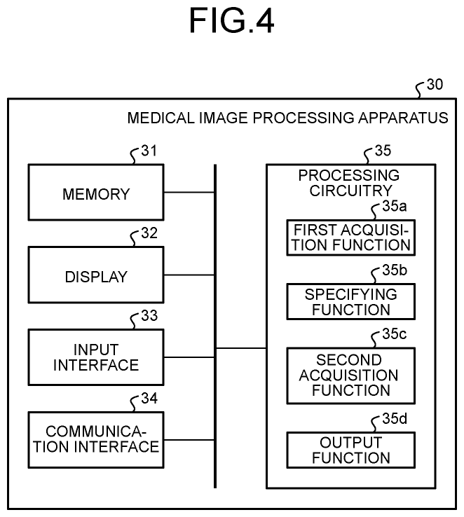

An example of the configuration of the medical image processing apparatus 30 is described below with reference to FIG. 4. FIG. 4 is a block diagram illustrating an example of the configuration of the medical image processing apparatus 30 according to the first embodiment. For example, the medical image processing apparatus 30 includes a memory 31, a display 32, an input interface 33, a communication interface 34, and processing circuitry 35. The medical image processing apparatus 30 is, for example, a workstation, a viewer, or the like.

Details of the memory 31, the display 32, the input interface 33, and the communication interface 34 are not particularly limited; however, for example, the memory 31, the display 32, the input interface 33, and the communication interface 34 can be configured in the same manner as the memory 125, the display 123, the input interface 122, and the communication interface 127 described above.

The processing circuitry 35 controls the overall operation of the medical image processing apparatus 30 by performing a first acquisition function 35a, a specifying function 35b, a second acquisition function 35c, and an output function 35d. For example, the processing circuitry 35 serves as the first acquisition function 35a by reading a computer program corresponding to the first acquisition function 35a from the memory 31 and executing the read computer program. In the same manner, the processing circuitry 35 serves as the specifying function 35b, the second acquisition function 35c, and the output function 35d.

The first acquisition function 35a is an example of a first acquisition unit. The specifying function 35b is an example of a specifying unit. The second acquisition function 35c is an example of a second acquisition unit. The output function 35d is an example of an output unit. Details of the first acquisition function 35a, the specifying function 35b, the second acquisition function 35c, and the output function 35d are described below.

In the medical image processing apparatus 30 illustrated in FIG. 4, the processing functions are stored in the memory 31 in the form of computer programs executable by a computer. The processing circuitry 35 is a processor that reads the computer programs from the memory 31 and executes the read computer programs, thereby implementing functions corresponding to the executed computer programs. In other words, the processing circuitry 35 in the state of having read the computer programs has functions corresponding to the read computer programs.

In FIG. 4, the first acquisition function 35a, the specifying function 35b, the second acquisition function 35c, and the output function 35d are described as being implemented by the single processing circuitry 35; however, the processing circuitry 35 may be configured by combining a plurality of independent processors and respective processors may implement functions by executing computer programs. Furthermore, the respective processing functions of the processing circuitry 35 may be implemented by being appropriately distributed or integrated into single processing circuitry or a plurality of pieces of processing circuitry.

The processing circuitry 35 may also implement functions by using a processor of an external device connected via the network NW. For example, the processing circuitry 35 reads computer programs corresponding to respective functions from the memory 31, executes the read computer programs, and uses, as computing resources, a group of servers (cloud) connected to the medical image processing apparatus 30 via the network NW, thereby implementing the respective functions illustrated in FIG. 4.

So far, the configuration example of the medical image diagnostic system 1 has been described. A region of interest is described below. The region of interest, for example, is a region that the user such as a physician pays attention to, such as a region including a suspected lesion or a treatment target region.

For example, before or during surgery, a three-dimensional blood vessel image is acquired by the X-ray diagnostic apparatus 10, and an index related to blood flow is acquired. For example, the X-ray diagnostic apparatus 10 rotates the C-arm 115 around the subject P to acquire a plurality of vessel images taken at different angles. For example, the X-ray diagnostic apparatus 10 is a biplane apparatus illustrated in FIG. 2B and substantially simultaneously acquires a plurality of blood vessel images taken at different angles. Then, the X-ray diagnostic apparatus 10 generates a three-dimensional blood vessel image from the plurality of blood vessel images taken at different angles, by using the principle of a stereo camera or the like.

A myocardial blood flow reserve ratio (fractional flow reserve (FFR)) is described below as an example of an index related to blood flow. The FFR can be measured, for example, by inserting a pressure wire into the blood vessel of the subject P.

Alternatively, the FFR can also be calculated on the basis of blood vessel images acquired by the X-ray diagnostic apparatus 10. For example, the biplane apparatus illustrated in FIG. 2B allows the acquisition of time-series blood vessel images from two directions. In this case, a time-series three-dimensional blood vessel image can be generated. Such a time-series three-dimensional blood vessel image shows the flow of a contrast agent in the blood vessels, and can be used to acquire fluid indices such as blood flow rate and flow velocity, for example. By performing fluid analysis on the basis of such fluid indices, the FFR at each position in the blood vessel can be calculated. Specifically, by the fluid analysis, pressure at a proximal portion close to the coronary artery and pressure at a distal portion far from the coronary artery can be calculated, and the FFR can be calculated by the formula “FFR=Pd (pressure at the distal portion)/Pa (pressure at the proximal portion)”.

The measured or calculated FFR can be tied to each position in the blood vessel image acquired by the X-ray diagnostic apparatus 10 and used as distribution data. On the basis of the distribution of the FFR, the user such as a physician can determine the condition of each position in the blood vessel and whether treatment is necessary. For example, a position with the FFR of “0.75” or less is subject to PCI treatment as a stenosis site, and is subject to a procedure for inserting a stent. Such a stent insertion region is an example of a region of interest.

A region with the FFR of “0.75 to 0.80” may not be candidates for PCI treatment, but rather for a follow-up observation, although a lesion is suspected. Such a region to be subject to a follow-up observation is also referred to as a defer region. The defer region is an example of a region of interest. The following is an example of the defer region where the FFR is “0.75 to 0.80”; however, specific numerical values are not limited to the above values and may be changed at the user's discretion.

After the PCI treatment, blood vessel images of the subject P are acquired by, for example, the X-ray CT apparatus 20 in order to perform a follow-up observation such as evaluation of treatment effects. For example, the X-ray CT apparatus 20 acquires X-ray CT images of the heart including coronary arteries while injecting a contrast agent. The user such as a physician interprets the X-ray CT images and records the interpreted results in an electronic medical record.

For example, the user first observes the volume of the entire heart including the coronary arteries. For example, the output function 244c generates a volume rendering image on the basis of the X-ray CT images and displays the volume rendering image on the display 242.

Next, the user checks the coronary arteries slice by slice. In particular, the user observes the presence or absence of plaques, changes in the FFR, and the like for slices including regions of interest such as the stent insertion region and the defer region described above. For example, by observing changes in the FFR for the stent insertion region, treatment effects can be evaluated. By observing changes in the FFR for the defer region, the progress of disease and the necessity of PCI treatment can be evaluated.

However, time and effort are required to observe the regions of interest such as the stent insertion region and the defer region. Specifically, the region of interest is set on the blood vessel images acquired by the X-ray diagnostic apparatus 10, for example, before or during PCI treatment. In order to find such regions of interest on the blood vessel images acquired by the X-ray CT apparatus 20 during another examination such as a follow-up observation, a plurality of slices generated from the X-ray CT images (volume data) need to be referred to. That is, before starting observation of the region of interest, the user may have to search for the region of interest.

In this regard, the medical image processing apparatus 30 in the medical image diagnostic system 1 facilitates comparison of regions of interest between blood vessel images acquired in different examinations by the processing of the processing circuitry 35 to be described in detail below.

The following describes a case where a first examination and a second examination after the first examination are performed. The first examination is, for example, a preoperative diagnosis performed using the blood vessel images acquired by the X-ray diagnostic apparatus 10, a PCI treatment performed using the blood vessel images acquired by the X-ray diagnostic apparatus 10, or the like. The second examination is, for example, a postoperative follow-up observation performed using the blood vessel images acquired by the X-ray CT apparatus 20. Blood vessel images acquired in the first examination are referred to as first blood vessel images, and blood vessel images acquired in the second examination are referred to as second blood vessel images.

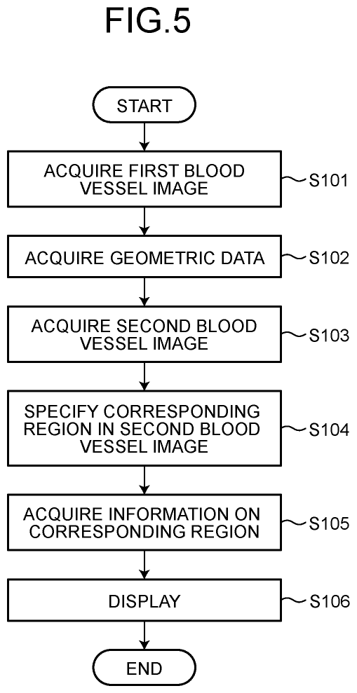

A process performed by the processing circuitry 35 of the medical image processing apparatus 30 is described according to a flowchart of FIG. 5.

First, the first acquisition function 35a acquires a first blood vessel image (step S101). For example, the first acquisition function 35a acquires, as the first blood vessel image, a three-dimensional blood vessel image taken by the X-ray diagnostic apparatus 10 before or during surgery. Subsequently, the first acquisition function 35a acquires geometric data indicating the position of a region of interest set on the first blood vessel image (step S102). The acquired geometric data is stored in the memory 31, for example.

For example, the first blood vessel image is taken before surgery, and a user who reads the first blood vessel image sets a region of interest such as a stent insertion region and a defer region on the first blood vessel image. For example, the first acquisition function 35a acquires the distribution of FFR in the first blood vessel image and sets a region where the FFR has a predetermined value as the region of interest. For example, the first acquisition function 35a sets a region where the FFR is “0.75 to 0.80” as the defer region. For example, the first blood vessel image is taken during a PCI treatment, and the first acquisition function 35a specifies the position of a stent by performing pattern matching on the first blood vessel image and sets a stent insertion region.

The geometric data indicating the position of the region of interest is, for example, data that ties position information of the region of interest to the first blood vessel image. For example, when the region of interest has been set by the user, the first acquisition function 35a can acquire geometric data indicating the position of the region of interest by acquiring the first blood vessel image. The first acquisition function 35a can also acquire geometric data indicating the position of the region of interest by setting a region where the FFR has a predetermined value as the defer region or by setting the stent insertion region by pattern matching.

In another example, the geometric data indicating the position of the region of interest is a mask image based on the first blood vessel image. For example, the mask image is a binarized image that is generated on the basis of the first blood vessel image and distinguishes pixel values of the region of interest from pixel values of a region other than the region of interest. As an example, a binarized image in which the pixel values of the region of interest in the first blood vessel image are set to “1” and the pixel values of a region other than the region of interest are set to “0” can be used as the mask image. The first acquisition function 35a may also generate the mask image on the basis of data that ties the position information of the region of interest to the first blood vessel image, or acquire a mask image generated by another device such as the X-ray diagnostic apparatus 10 via the network NW.

In another example, the geometric data indicating the position of the region of interest may be text data indicating position coordinates of the region of interest. For example, the text data is a position vector (x, y, z) indicating the orientation and length from the stent insertion region to the defer region. The first acquisition function 35a may also generate text data on the basis of data that ties the position information of the region of interest to the first blood vessel image, or acquire text data generated by another device such as the X-ray diagnostic apparatus 10 via the network NW.

Subsequently, the first acquisition function 35a acquires a second blood vessel image (step S103). For example, the first acquisition function 35a acquires, as the second blood vessel image, a three-dimensional blood vessel image taken by the X-ray CT apparatus 20 during a postoperative follow-up observation.

At step S101, the first acquisition function 35a may also acquire the first blood vessel image directly from the X-ray diagnostic apparatus 10 or via another device such as an image storage device. Similarly, at step S103, the first acquisition function 35a may also acquire the second blood vessel image directly from the X-ray CT apparatus 20 or via another device such as an image storage device. An example of such an image storage device may include a picture archiving and communication system (PACS) server.

Subsequently, on the basis of the geometric data indicating the position of the region of interest, the specifying function 35b specifies a corresponding region corresponding to the region of interest in the second blood vessel image (step S104).

For example, the first acquisition function 35a acquires, as the geometric data indicating the position of the region of interest, data that ties the position information of the region of interest to the first blood vessel image. In this case, the specifying function 35b aligns the first blood vessel image with the second blood vessel image. For example, the specifying function 35b performs non-rigid alignment between the first blood vessel image and the second blood vessel image on the basis of blood vessel shapes or anatomical feature points such as bone or soft tissue. This allows the specifying function 35b to specify a corresponding region in the second blood vessel image that corresponds to the region of interest set on the first blood vessel image.

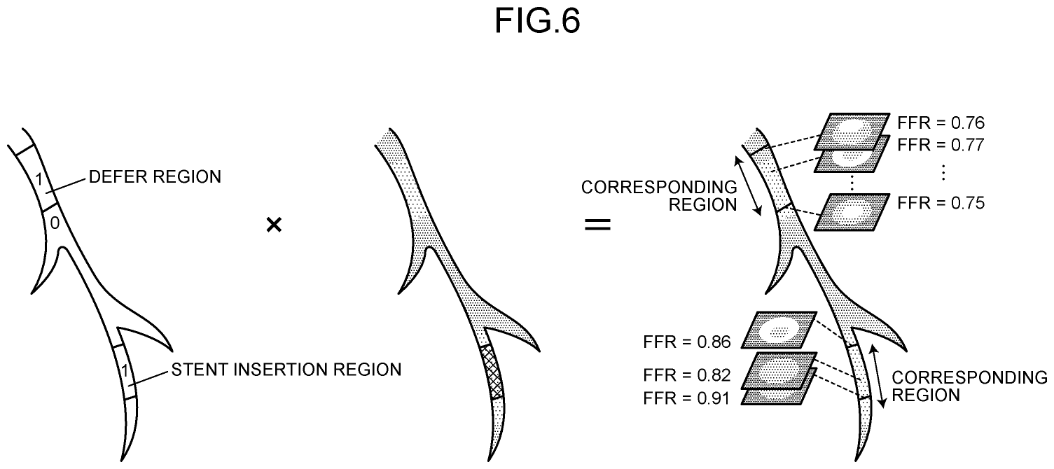

For example, the first acquisition function 35a acquires a mask image based on the first blood vessel image as the geometric data indicating the position of the region of interest. The specifying of the corresponding region based on the mask image is described below with reference to FIG. 6.

The left figure of FIG. 6 is an example of the mask image. Specifically, the stent insertion region and the defer region in the coronary artery are defined as regions of interest, and a pixel value is set to “1”. Pixel values of regions other than the regions of interest are set to “0”. As illustrated in the middle figure of FIG. 6, the specifying function 35b specifies the stent insertion region from the second blood vessel image by pattern matching or the like. Subsequently, the specifying function 35b aligns the mask image with the second blood vessel image so that the stent insertion region indicated by the mask image matches the stent insertion region specified in the second blood vessel image. This allows the specifying function 35b to specify a corresponding region in the second blood vessel image that corresponds to the region of interest set on the first blood vessel image.

For example, the first acquisition function 35a acquires text data indicating position coordinates of the region of interest, as the geometric data indicating the position of the region of interest. For example, the first acquisition function 35a acquires a position vector (x, y, z) indicating the orientation and length from the stent insertion region to the defer region. In this case, the specifying function 35b specifies the stent insertion region, for example, from the second blood vessel image by pattern matching or the like. Subsequently, by adding the position vector (x, y, z) to the position coordinates of the specified stent insertion region, the specifying function 35b can specify a corresponding region in the second blood vessel image that corresponds to the region of interest set on the first blood vessel image.

Subsequently, the second acquisition function 35c acquires information on the corresponding region specified by the specifying function 35b (step S105).

For example, the second acquisition function 35c acquires, as information on the corresponding region, a two-dimensional image including the corresponding region from the second blood vessel image. For example, the second acquisition function 35c acquires a slice orthogonal to a core line for each position in a blood vessel region specified as the corresponding region in the coronary artery. That is, the second acquisition function 35c acquires circular slices of the coronary artery.

The two-dimensional image to be acquired as the information on the corresponding region can be arbitrarily transformed. For example, the second acquisition function 35c may acquire a curved planer reconstruction (CPR) image of the coronary artery that includes the core line of the blood vessel region specified as the corresponding region. For example, the second acquisition function 35c may also acquire slices in a predetermined direction that include the corresponding region. In one example, the second acquisition function 35c acquires slices that include the corresponding region and are in the axial, coronal, and sagittal planes, respectively.

For example, the second acquisition function 35c acquires an index related to blood flow in the corresponding region as the information on the corresponding region. For example, the second acquisition function 35c acquires FFR for each position in the blood vessel region specified as the corresponding region in the coronary artery. For example, the second acquisition function 35c acquires FFR measured by a pressure wire on the same day as the X-ray CT apparatus 20 acquires the second blood vessel image. Alternatively, the second acquisition function 35c acquires FFR calculated on the basis of the second blood vessel image.

Subsequently, the output function 35d displays the information on the corresponding region (step S106). For example, as illustrated in the right figure of FIG. 6, the output function 35d displays circular slices of the coronary artery for each position in the corresponding region, along with the FFR values for each position.

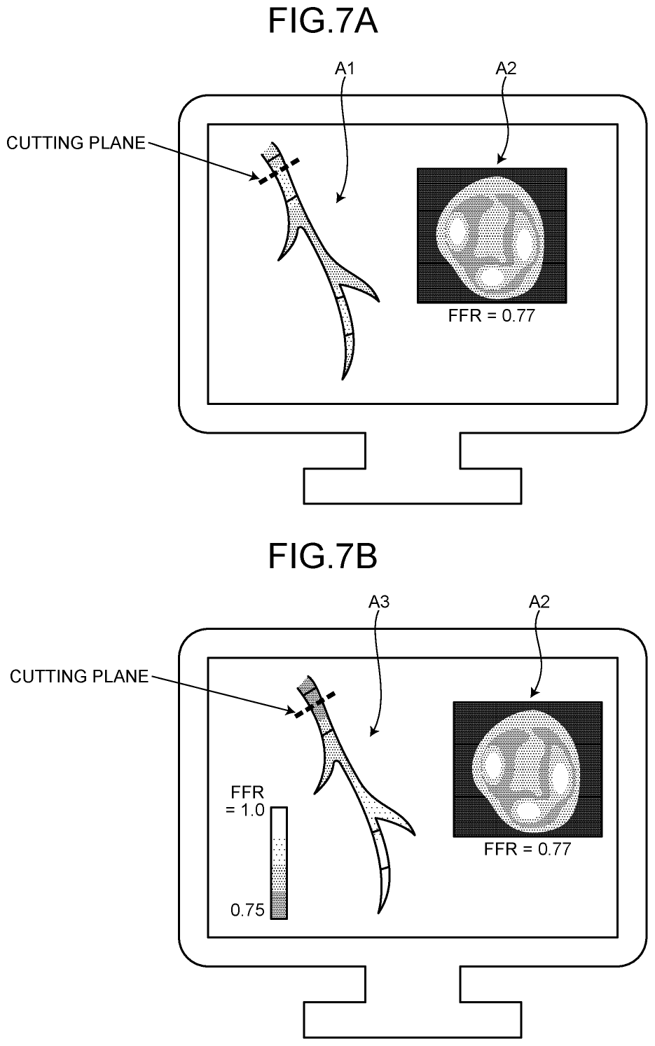

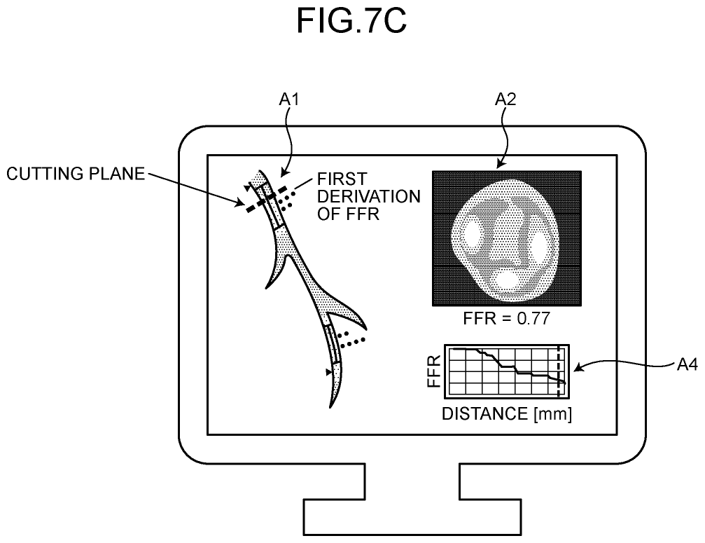

Variations of the display at step S106 are illustrated in FIGS. 7A to 7C. For example, as illustrated in FIG. 7A, the output function 35d displays a two-dimensional image A1 and a two-dimensional image A2 side by side, including the corresponding region. The two-dimensional image A1 is a CPR image along the core line of the coronary artery. The two-dimensional image A2 is a circular slice of the coronary artery. The output function 35d may display a cutting plane of the two-dimensional image A1 that corresponds to the position of the two-dimensional image A2. The output function 35d may also display the FFR value at the position of the two-dimensional image A2. That is, the output function 35d may display the FFR value in the corresponding region.

According to FIG. 7A, the user such as a physician can evaluate the degree of calcification and plaque attachment for the stent insertion region and the defer region. The output function 35d may receive input operations from the user on the two-dimensional image A1, move the position of the cutting plane, and sequentially change the two-dimensional image A2 according to the moved position.

FIG. 7B is an example of displaying a color image A3 instead of the two-dimensional image A1 in FIG. 7A. The color image A3 is an image obtained by assigning a color according to the FFR value to the CPR image along the core line of the coronary artery. FIG. 7B is an example illustrating a graph A4 and a first derivation of the FFR, as opposed to the display in FIG. 7A. In the graph A4, for example, a horizontal axis denotes the distance along the core line with a proximal portion as a reference and a vertical axis denotes FFR values at each position along the core line. The first derivation in the graph A4 represents the amount of change in the FFR at a position in the core line of the coronary artery, and when a plurality of positions with a large amount of change are present, treatment may be started from a location with the largest amount of change. Thus, the order of treatment priority may be visualized as illustrated in FIG. 7B.

In FIG. 5, an example of acquiring and displaying the information on the corresponding region is described; however, the embodiment is not limited to this example. For example, after the information on the corresponding region is acquired, the information on the corresponding region may also be stored in the memory 31 instead of or in addition to the display by the output function 35d.

For example, the second acquisition function 35c may automatically store, in the electronic medical record, a two-dimensional image including the corresponding region generated on the basis of the second blood vessel image and the FFR values in the corresponding region. In one example, as illustrated in the right figure of FIG. 6, the second acquisition function 35c acquires a plurality of circular slices of the coronary artery for each position in the corresponding region. Subsequently, the second acquisition function 35c evaluates the amount of calcification and plaque for each of the plurality of acquired slices. Subsequently, the second acquisition function 35c selects a slice with many calcifications and plaques, stores the selected slice in the electronic medical record, and stores an FFR value at the position of the stored slice.

The second acquisition function 35c may also store the FFR value as numerical data or in text form. For example, the second acquisition function 35c automatically generates a sentence “FFR value in slice XX is YY” and stores the sentence in the electronic medical record.

As described above, the first acquisition function 35a of the embodiment acquires the geometric data indicating the position of the region of interest set on the first blood vessel image. The specifying function 35b specifies the corresponding region corresponding to the region of interest in the second blood vessel image on the basis of the geometric data. The second acquisition function 35c acquires the information on the corresponding region. This allows the medical image processing apparatus 30 of the embodiment to facilitate comparison of regions of interest between blood vessel images acquired during different examinations.

For example, before or during a PCI treatment, the region of interest such as the stent insertion region or the defer region is set on the first blood vessel image by the X-ray diagnostic apparatus 10. In a subsequent follow-up observation, the second blood vessel image is acquired by the X-ray CT apparatus 20. According to the medical image processing apparatus 30 of the embodiment, the corresponding region corresponding to the region of interest can be automatically specified, and a two-dimensional image including, for example, the stent insertion region or the defer region and the FFR value at that position can also be further automatically presented. This reduces a workload of a user in the follow-up observation using the second blood vessel image.

In addition to the first embodiment described above, various variations may be implemented.

For example, FIG. 1 describes a case where the single medical image processing apparatus 30 is connected to the X-ray diagnostic apparatus 10 and the X-ray CT apparatus 20; however, the medical image processing apparatus 30 may be implemented by combining a plurality of apparatuses. For example, the medical image processing apparatus 30 is implemented by combining a first medical image processing apparatus and a second medical image processing apparatus. The first medical image processing apparatus is connected to, for example, the X-ray diagnostic apparatus 10, and acquires the geometric data indicating the position of the region of interest set on the first blood vessel image. The second medical image processing apparatus is connected to, for example, the X-ray CT apparatus 20, and specifies the corresponding region corresponding to the region of interest in the second blood vessel image on the basis of the geometric data, and acquires the information on the specified corresponding region.

For example, FIG. 1 describes a case where the medical image processing apparatus 30 is provided separately from the X-ray diagnostic apparatus 10 and the X-ray CT apparatus 20; however, the medical image processing apparatus 30 may be implemented as a part of the X-ray diagnostic apparatus 10 or the X-ray CT apparatus 20. For example, the first acquisition function 35a, the specifying function 35b, the second acquisition function 35c, and the output function 35d described above may be performed by the processing circuitry 244 in the X-ray CT apparatus 20. In this case, the console 240 is an example of the medical image processing apparatus 30.

In the embodiment described above, the first blood vessel image acquired by the X-ray diagnostic apparatus 10 before or during surgery and the second blood vessel image acquired by the X-ray CT apparatus 20 during a follow-up observation are described. However, the embodiment is not limited to this configuration.

For example, the first blood vessel image may be an X-ray CT image acquired by the X-ray CT apparatus 20. That is, the first blood vessel image and the second blood vessel image may be acquired by the same medical image diagnostic apparatus.

For example, the first blood vessel image may be a blood vessel image acquired in a follow-up observation, and the second blood vessel image may be another blood vessel image acquired in a subsequent follow-up observation. In this case, the first acquisition function 35a acquires the geometric data indicating the position of the region of interest set on the first blood vessel image. The specifying function 35b specifies the corresponding region corresponding to the region of interest in the second blood vessel image on the basis of the geometric data. The second acquisition function 35c acquires the information on the corresponding region. On the basis of the information on the corresponding region, the first acquisition function 35a can set a new region of interest. For example, on the basis of an FFR value calculated on the basis of the second blood vessel image, the first acquisition function 35a may set a new defer.

In the embodiment described above, a case where the first blood vessel image and the second blood vessel image are acquired for the coronary artery is described; however, the same can also be applied to other sites such as carotid arteries and cerebral vessels, for example.

Although the description is based on an example of an examination related to angiostenosis, the same can also be applied to examinations for different purposes. For example, in order to prevent rupture of blood vessels such as aneurysms, a procedure of inserting a stent may be performed. In a follow-up observation after such a procedure, the above embodiment may be applied by setting a region of interest based on values of parameters such as blood flow and stress on vessel walls, in addition to a region where the stent is inserted.

In the embodiment described above, the FFR is described as an example of the index related to blood flow; however, the embodiment is not limited to the FFR. For example, the index related to blood flow may employ a coronary flow reserve (CFR), an instant wave-free ratio (iFR), an index of microcirculatory resistance (IMR), a wall shear stress (WSS), an oscillatory shear index (OSI) representing fluctuation of WSS, or the like.

The term “processor” used in the above description, for example, means circuitry such as a CPU, a graphics processing unit (GPU), an application specific integrated circuit (ASIC), or a programmable logic device (for example, a simple programmable logic device (SPLD), a complex programmable logic device (CPLD), and a field programmable gate array (FPGA)). When the processor is, for example, a CPU, the processor implements functions by reading and executing computer programs stored in storage circuitry. On the other hand, when the processor is, for example, an ASIC, a corresponding function is directly incorporated in the circuitry of the processor as logic circuitry instead of storing the computer programs in the storage circuitry. Each processor of the embodiment is not limited to being configured as a single piece of circuitry for each processor, and one processor may be configured by combining a plurality of pieces of independent circuitry to implement the functions thereof. The plurality of components in each diagram may be integrated into one processor to implement the functions thereof.

In the above description, a single memory stores a computer program corresponding to each processing function of the processing circuitry. However, the embodiment is not limited to this configuration. For example, a plurality of memories may be arranged in a distributed manner and the processing circuitry may be configured to read corresponding programs from the individual memories. Instead of storing the computer programs in the memory, the computer programs may be directly incorporated in the circuitry of the processor. In this case, the processor implements functions by reading and executing the computer programs incorporated in the circuitry.

Each component of each apparatus according to the embodiment described above is a functional conceptual, and does not necessarily have to be physically configured as illustrated in the drawings. That is, the specific form of distribution or integration of each apparatus is not limited to those illustrated in the drawings, and can be configured by functionally or physically distributing or integrating all or part thereof in arbitrary units, depending on various loads and use conditions. Moreover, each processing function performed by each apparatus can be implemented in whole or in part by a CPU and a computer program that is analyzed and executed by the CPU, or by hardware using wired logic.

The medical image processing method described in the embodiment described above can be implemented by executing a computer program prepared in advance on a computer such as a personal computer or a workstation. The computer program can be distributed via a network such as the Internet. The computer program can also be recorded on a non-transitory computer-readable recording medium such as a hard disk, a flexible disk (FD), a CD-ROM, an MO, and a DVD, and can be executed by being read from the recording medium by a computer.

According to at least one embodiment described above, comparison of regions of interest between blood vessel images acquired during different examinations can be facilitated.

While certain embodiments have been described, these embodiments have been presented by way of example only, and are not intended to limit the scope of the inventions. Indeed, the novel embodiments described herein may be embodied in a variety of other forms; furthermore, various omissions, substitutions and changes in the form of the embodiments described herein may be made without departing from the spirit of the inventions. The accompanying claims and their equivalents are intended to cover such forms or modifications as would fall within the scope and spirit of the inventions.

What is claimed is:

1. A medical image processing apparatus comprising processing circuitry configured to
acquire geometric data indicating a position of a region of interest set on a first blood vessel image,
specify a corresponding region in a second blood vessel image that corresponds to the region of interest on a basis of the geometric data, and
acquire information on the corresponding region.

2. The medical image processing apparatus according to claim 1, wherein the information on the corresponding region is an index related to blood flow in the corresponding region.

3. The medical image processing apparatus according to claim 1, wherein the information on the corresponding region is a slice including the corresponding region in the second blood vessel image.

4. The medical image processing apparatus according to claim 1, wherein the geometric data is data that ties position information of the region of interest to the first blood vessel image.

5. The medical image processing apparatus according to claim 1, wherein the geometric data is a binarized image that is generated on a basis of the first blood vessel image and distinguishes pixel values of the region of interest from pixel values of a region other than the region of interest.

6. The medical image processing apparatus according to claim 1, wherein the geometric data is text data indicating position coordinates of the region of interest.

7. The medical image processing apparatus according to claim 1, wherein the processing circuitry further allows the information on the corresponding region to be displayed.

8. The medical image processing apparatus according to claim 1, wherein the processing circuitry stores the information on the corresponding region in a memory.

9. The medical image processing apparatus according to claim 1, wherein the first blood vessel image is a blood vessel image acquired by an X-ray angiography apparatus and the second blood vessel image is a blood vessel image acquired by an X-ray CT apparatus.

10. The medical image processing apparatus according to claim 2, wherein the index related to blood flow is based on output acquired by inserting a pressure wire into a blood vessel of subject.

11. A non-transitory computer readable medium for storing computer program, wherein the computer program causes a computer to perform a method comprising:
acquiring geometric data indicating a position of a region of interest set on a first blood vessel image,
specifying a corresponding region in a second blood vessel image that corresponds to the region of interest on a basis of the geometric data, and
acquiring information on the corresponding region.

12. The non-transitory computer readable medium according to claim 11, wherein the information on the corresponding region is an index related to blood flow in the corresponding region.

13. The non-transitory computer readable medium according to claim 11, wherein the information on the corresponding region is a slice including the corresponding region in the second blood vessel image.

14. The non-transitory computer readable medium according to claim 11, wherein the geometric data is data that ties position information of the region of interest to the first blood vessel image.

15. The non-transitory computer readable medium according to claim 11, wherein the geometric data is a binarized image that is generated on a basis of the first blood vessel image and distinguishes pixel values of the region of interest from pixel values of a region other than the region of interest.

16. The non-transitory computer readable medium according to claim 11, wherein the geometric data is text data indicating position coordinates of the region of interest.

17. The non-transitory computer readable medium according to claim 11 further storing computer program causes a computer to perform a method comprising allowing the information on the corresponding region to be displayed.

18. The non-transitory computer readable medium according to claim 11 further storing computer program causes a computer to perform a method comprising storing the information on the corresponding region in a memory.

19. The non-transitory computer readable medium according to claim 11, wherein the first blood vessel image is a blood vessel image acquired by an X-ray angiography apparatus and the second blood vessel image is a blood vessel image acquired by an X-ray CT apparatus.

20. The non-transitory computer readable medium according to claim 12, wherein the index related to blood flow is based on output acquired by inserting a pressure wire into a blood vessel of subject.
